(19) United States
(12) Patent Application Publication
(10) Pub. No.: US 2024/0090838 A1
(43) Pub. Date: Mar. 21, 2024

(54) PHYSIOLOGICAL SIGNAL MONITORING DEVICE AND METHOD FOR ENHANCING ADHESION THEREOF

(71) Applicant: BIONIME CORPORATION, Taichung City (TW)

(72) Inventors: Chieh-Hsing CHEN, Taichung City (TW); Kuan-Lin CHANG, Taichung City (TW)

(21) Appl. No.: 18/155,755

(22) Filed: Jan. 18, 2023

(30) Foreign Application Priority Data
Sep. 15, 2022 (TW) 111134898

(51) Int. Cl. A61B 5/00; A61B 5/145

(52) U.S. Cl. CPC A61B 5/6833; A61B 5/14532

(57) ABSTRACT

A physiological signal monitoring device includes a base, a sensor, a transmitter, an adhesive layer and a pad. The sensor is carried by the base. The transmitter is coupled to the sensor. The adhesive layer is arranged on a bottom surface of the base. The pad includes an adhesive backing and a coupling backing. The adhesive backing is fabricated by weaving first threads and includes first holes. The coupling backing provides a coupling surface, and is fabricated by weaving second threads and includes second holes and piques. The piques are arranged on the coupling surface to form convex and concave three-dimensional textures on the coupling surface. The adhesive layer soaks the pad through the second holes and wraps at least one of the second threads and the first threads, so as to make the pad be connected to the base through the adhesive layer.

### RELATED APPLICATIONS

This application claims priority to Taiwan Application Serial Number 111134898, filed Sep. 15, 2022, which is herein incorporated by reference.

### BACKGROUND

### Technical Field

The present disclosure relates to a physiological signal monitoring device and a method for enhancing an adhesion thereof. More particularly, the present disclosure relates to a physiological signal monitoring device and a method for enhancing an adhesion thereof which can perform both air permeability and connecting strength.

### Description of Related Art

To the patients suffering from chronic diseases, it is an important key to control diseases by monitoring the physiological signals of one's own periodically and regularly. Take diabetes for example, it is better for patients suffering from diabetes to check blood sugar more than three times a day, and each time a lancet is used to obtain the patient's blood for testing. Therefore, in order to make the patients receive fewer pricks, the continuous glucose monitoring (CGM) system is developed. The continuous glucose monitoring system usually includes a base, a sensor and a transmitter. The sensor can be implanted by an insertion needle and stay under the skin of the patient for a long time, so as to measure the glucose in the interstitial fluid to obtain a physiological signal. The transmitter is able to receive and transmit the physiological signal. Both the transmitter and the sensor can be installed to the skin surface of the patient through the base and a patch.

The user has to wear the continuous glucose monitoring system for a long time. Moisture or sweat is prone to accumulating between the skin surface and the base due to the long time wear, which affects the performance of adhesion therebetween. Therefore, the problem of air permeability between the base and the skin surface becomes important. Reference is made to FIG. 9. FIG. 9 is a sectional schematic view of a patch 950 and a base 910 of a conventional physiological signal monitoring device 900. The conventional physiological signal monitoring device 900, such as the continuous glucose monitoring system, should be attached to the skin surface of the patient for a long time. The patch 950 of the conventional physiological signal monitoring device 900 is designed to solve the aforementioned problem by having a plurality of through holes 951 which are vertically extended therethrough. However, moisture or sweat is still prone to accumulating between the skin surface and the base 910 or within the through holes 951 after the long time wear, which makes the patient feel uncomfortable. Also, the adhesion between the patch 950 and the skin surface or the base 910 is possibly decreased, which causes the problems such as the conventional physiological signal monitoring device 900 is easy to fall off prematurely because it is caught as the user putting on or taking off clothes or because of hitting.

In this regard, it is still an unsolved problem to maintain air permeability between the base and the skin surface and connecting strength between the base and the patch at the same time.

### SUMMARY

According to one embodiment of one aspect of the present disclosure, a physiological signal monitoring device, which is used for monitoring at least one analyte in an organism, includes a base, a sensor, a transmitter, an adhesive layer and a pad. The base is configured to be arranged on a skin surface of the organism, and the base includes a bottom surface. The sensor is carried by the base, wherein the sensor is at least partially implanted under the skin surface of the organism, so as to measure and output a physiological signal corresponding to the analyte. The transmitter is detachably disposed on the base, wherein the transmitter is coupled to the sensor for receiving and transmitting the physiological signal. The adhesive layer is arranged on the bottom surface of the base. The pad includes an adhesive backing and a coupling backing. The adhesive backing provides an adhesive surface, wherein the adhesive backing is fabricated by weaving a plurality of first threads and includes a plurality of first holes. The coupling backing provides a coupling surface, wherein the coupling backing is stacked on the adhesive backing and fabricated by weaving a plurality of second threads to include a plurality of second holes and a plurality of piques. The plurality of second holes are at least open at the coupling surface, and the plurality of piques are arranged on the coupling surface to form convex and concave three-dimensional textures on the coupling surface. The pad is attached to the skin surface of the organism through the adhesive surface, and at least part of the adhesive layer soaks the pad through the plurality of second holes of the coupling backing and at least partially wraps at least one of the plurality of second threads and the plurality of first threads, so as to make the pad be connected to the base through the adhesive layer.

According to another embodiment of the one aspect of the present disclosure, a physiological signal monitoring device, which is used for monitoring at least one analyte in an organism, includes a base, a sensor, a transmitter, an adhesive layer and a pad. The base is configured to be arranged on a skin surface of the organism, and the base includes a bottom surface. The sensor is carried by the base, wherein the sensor is at least partially implanted under the skin surface of the organism, so as to measure and output a physiological signal corresponding to the analyte. The transmitter is detachably disposed on the base, wherein the transmitter is coupled to the sensor for receiving and transmitting the physiological signal. The adhesive layer is arranged on the bottom surface of the base. The pad at least includes an adhering layer and a coupling layer. The adhering layer provides an adhesive surface and includes a plurality of first holes, and the coupling layer provides a coupling surface and includes a plurality of second holes, which are open at the coupling surface. The pad is attached to the skin surface of the organism through the adhesive surface, and at least part of the adhesive layer soaks the pad through the coupling surface of the coupling layer, so as to make the pad be connected to the base through the adhesive layer.

According to an embodiment of another aspect of the present disclosure, a method for enhancing an adhesion of the physiological signal monitoring device of the aforementioned aspect on a skin surface of an organism includes the steps as follows. The base is provided, an adhesive-layer attaching step is performed, a pad attaching step is performed, a hot-pressing step is performed and an assembling step is performed. In the adhesive-layer attaching step, the adhesive layer is attached to the bottom surface of the base. In the pad attaching step, the pad is attached to the adhesive layer. The hot-pressing step is performed by hot pressing from the pad toward the base, so as to make the pad be connected to the base through the adhesive layer. In the assembling step, when a user uses the physiological signal monitoring device to monitor the analyte in the organism, the transmitter and the sensor are assembled on the base which has been hot pressed, so as to finish setting up the physiological signal monitoring device.

### BRIEF DESCRIPTION OF THE DRAWINGS

The present disclosure can be more fully understood by reading the following detailed description of the embodiment, with reference made to the accompanying drawings as follows:

FIG. 1 is a three-dimensional schematic view of a physiological signal monitoring device according to an embodiment of an aspect of the present disclosure.

FIG. 2 is an exploded schematic view of the physiological signal monitoring device of FIG. 1.

FIG. 3 is an exploded schematic view of a pad of the physiological signal monitoring device of FIG. 1.

FIG. 4 is a sectional schematic view along the line 4-4 of the physiological signal monitoring device of FIG. 1.

FIG. 5A is a sectional schematic view along the line 5A-5A of the pad of the physiological signal monitoring device of FIG. 2.

FIG. 5B is a partially enlarged schematic view of the region 5B of the physiological signal monitoring device of FIG. 4.

FIG. 6A is a step flow chart of a method for enhancing an adhesion of the physiological signal monitoring device on a skin surface of an organism according to an embodiment of another aspect of the present disclosure.

FIG. 6B is a step flow chart of the method for enhancing the adhesion of the physiological signal monitoring device on the skin surface of the organism according to another embodiment of another aspect of the present disclosure.

FIG. 7 is a test diagram showing the retaining force between the pad and the base of Comparative Example 1 and Example 1.

FIG. 8 is a simulating test diagram showing the adhesion between the pad and the skin surface of the organism of Example 2.

FIG. 9 is a sectional schematic view of a patch and a base of a conventional physiological signal monitoring device.

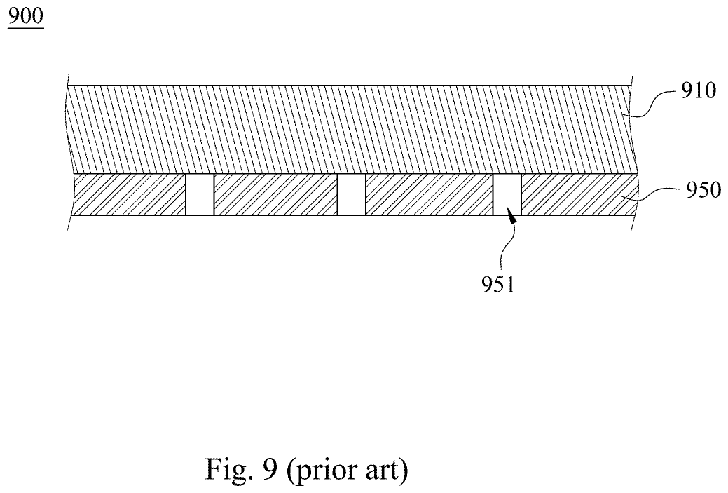

### DETAILED DESCRIPTION

The present disclosure will be further exemplified by the following specific embodiments. However, the embodiments can be applied to various inventive concepts and can be embodied in various specific ranges. The specific embodiments are only for the purposes of description, and are not limited to these practical details thereof. Furthermore, in order to simplify the drawings, some conventional structures and elements will be illustrated in the drawings by a simple and schematic way.

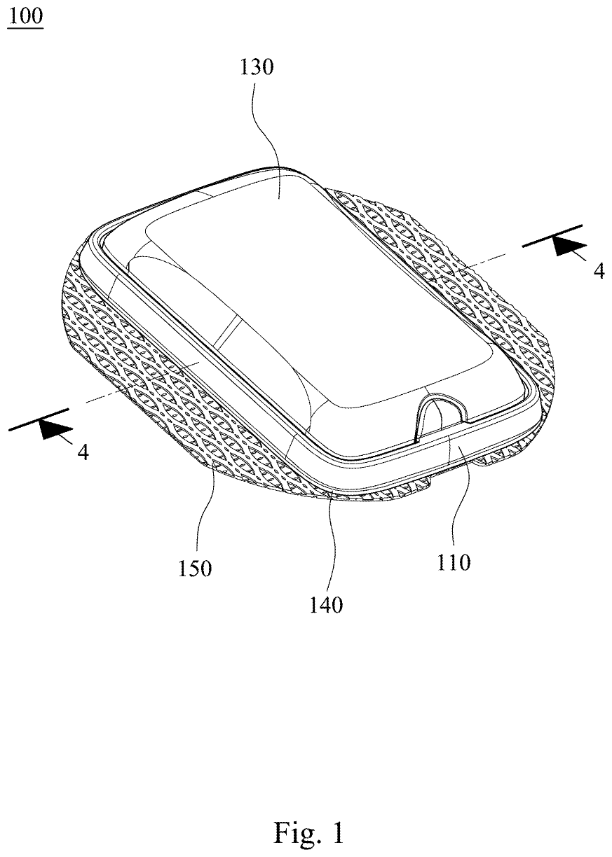

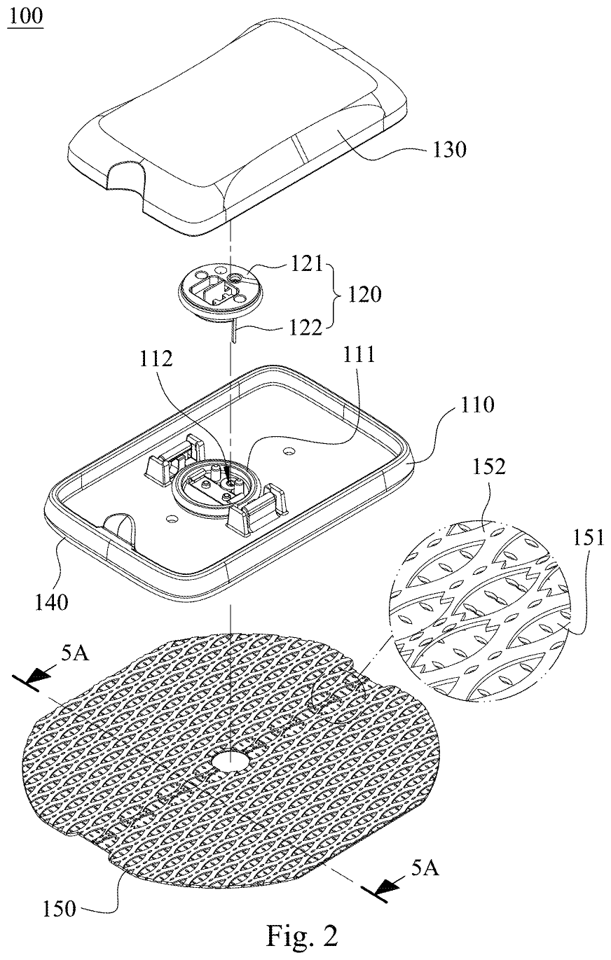

Reference is made to FIG. 1 and FIG. 2. FIG. 1 is a three-dimensional schematic view of a physiological signal monitoring device 100 according to an embodiment of an aspect of the present disclosure. FIG. 2 is an exploded schematic view of the physiological signal monitoring device 100 of FIG. 1. The physiological signal monitoring device 100 is used for monitoring at least one analyte in an organism. The physiological signal monitoring device 100 includes a base 110, a sensor 120, a transmitter 130, an adhesive layer 140 and a pad 150. The sensor 120, the transmitter 130 and the adhesive layer 140 are respectively connected to the base 110. The pad 150 is connected to the base 110 through the adhesive layer 140.

In detail, the base 110 is configured to be arranged on a skin surface of the organism, and the base 110 includes a bottom surface (the number is omitted). The sensor 120 is configured to measure the data of the analyte in the organism, and output a physiological signal corresponding to the data of the analyte to the transmitter 130. The sensor 120 is carried by the base 110 and at least partially implanted under the skin surface of the organism. For example, the sensor 120 includes a mounting seat 121, which is carried by the base 110, and a sensing member 122, which is disposed at the mounting seat 121 and limited to the mounting seat 121. The sensing member 122 is at least partially implanted under the skin surface of the organism, so as to measure and output the physiological signal corresponding to the analyte. In FIG. 2, the base 110 can include a supporting portion 111 and an insertion hole 112. The sensor 120 can be limited to the supporting portion 111, and the sensing member 122 is at least partially implanted under the skin surface of the organism through the insertion hole 112. In the present embodiment, the sensing member 122 is implanted under the skin surface of the organism through the insertion hole 112 with the leading of an insertion needle (not shown), and the present disclosure is not limited thereto.

The transmitter 130 is detachably disposed on the base 110. The transmitter 130 is coupled to the sensor 120 for receiving and transmitting the physiological signal. The transmitter 130 can include a circuit board (not shown), a connection port (not shown) or other electronic components (not shown). The aforementioned circuit board and electronic components can be disposed in the transmitter 130. Furthermore, a space can be delimited by the transmitter 130 and the base 110 for accommodating and positioning the sensor 120. Sealing structures, which respectively match the transmitter 130, the sensor 120 and the base 110, can be arranged. When the transmitter 130 is coupled to the base 110, the aforementioned sealing structures ensure that liquid does not penetrate therein along the path between the insertion hole 112, the transmitter 130 and the base 110. The risk of failures in the sensor 120 and the transmitter 130 due to moisture can be reduced, and the circuit board and other electronic components in the transmitter 130 can be well protected.

The adhesive layer 140 is arranged between the bottom surface of the base 110 and the pad 150, so as to help the connection of the base 110 and the pad 150. The material of the adhesive layer 140 can be any adhesive with biocompatibility, such as ethylene-vinyl acetate (EVA) type adhesive, thermoplastic rubber (TPR) type adhesive, polyurethane (PUR) type adhesive, silicone type adhesive or acrylic acid type adhesive. It should be noticed that, the adhesive layer 140 can only cover a portion of the bottom surface of the base 110. The area of the adhesive layer 140 on the bottom surface can be 25%-50%, preferably 25%-40%, more preferably 28%-35%, of the total area of the bottom surface. The air permeability among the skin surface of the organism, the pad 150, the base 110 and outside can be enhanced, and the influence on adhesion between the pad 150 and the skin surface of the organism can be prevented. For example, the bottom surface of the base 110 can be in a rectangular shape, and the adhesive layer 140 can be arranged at four corners of the bottom surface. Alternatively, the adhesive layer 140 can be arranged along a peripheral edge of the bottom surface, which makes the adhesive layer 140 be arranged on the bottom surface in a U shape. Therefore, a balance between the retaining force and air permeability between the pad 150 and the base 110 can be reached.

When the base 110 includes the insertion hole 112, and the sensing member 122 is at least partially implanted under the skin surface of the organism through the insertion hole 112, the adhesive layer 140 can be disposed around the insertion hole 112 to prevent the body fluid of the organism soaking the pad 150. It is favorable for maintaining the retaining force between the pad 150 and the base 110 or the adhesion between the pad 150 and the skin surface of the organism. At the same time, the adhesive layer 140 arranged around the insertion hole 112 can also prevent liquid from outside (such as sewage) penetrating through the pad 150 and touching the wound. Thus, the risk of wound infection can be reduced.

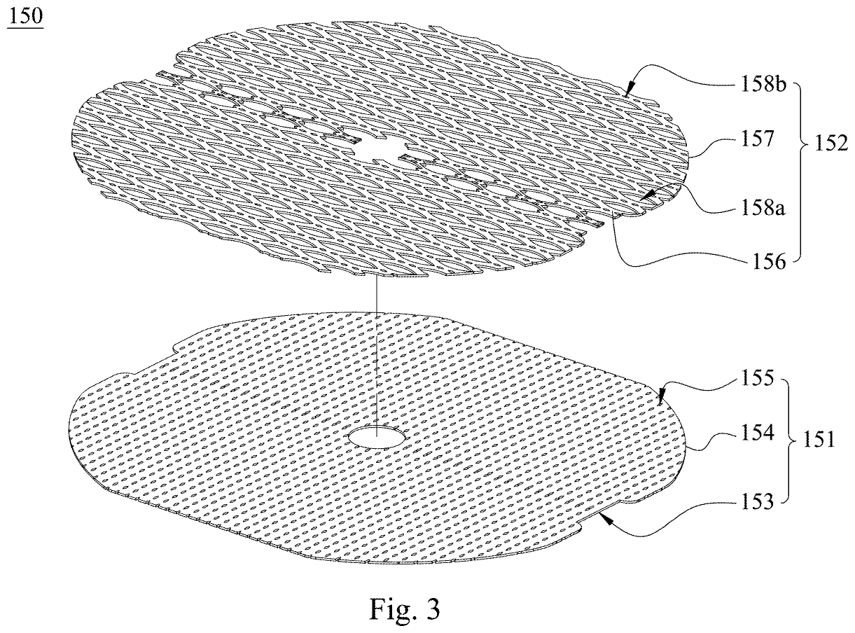

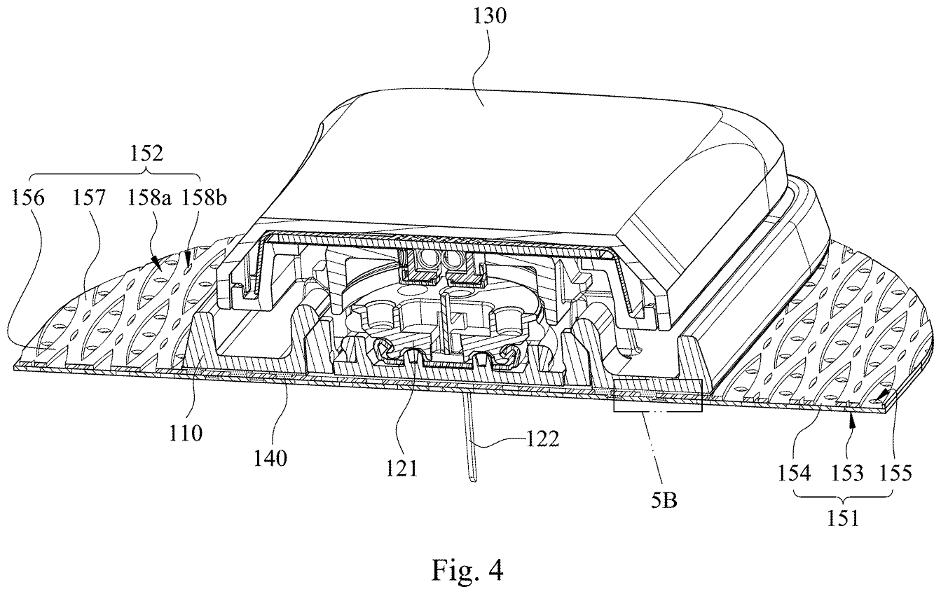

Reference is made to FIG. 3 and FIG. 4. FIG. 3 is an exploded schematic view of the pad 150 of the physiological signal monitoring device 100 of FIG. 1. FIG. 4 is a sectional schematic view along the line 4-4 of the physiological signal monitoring device 100 of FIG. 1. The pad 150 includes an adhesive backing 151 and a coupling backing 152, and the coupling backing 152 is stacked on the adhesive backing 151. The adhesive backing 151 provides an adhesive surface 153. The adhesive backing 151 is fabricated by weaving a plurality of first threads 154 to include a plurality of first holes 155. The pad 150 is attached to the skin surface of the organism through the adhesive surface 153. The coupling backing 152 provides a coupling surface 156. The coupling backing 152 is fabricated by weaving a plurality of second threads 157 to include a plurality of second holes 158a, which are at least open at the coupling surface 156.

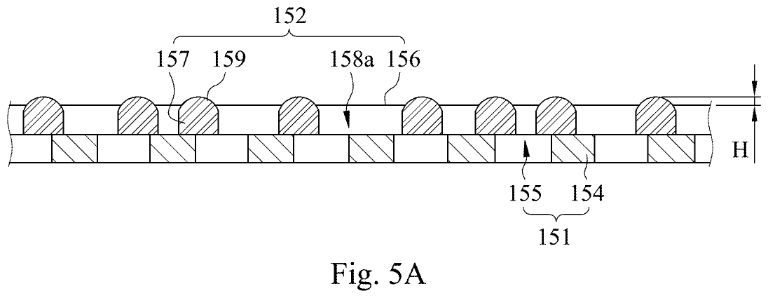

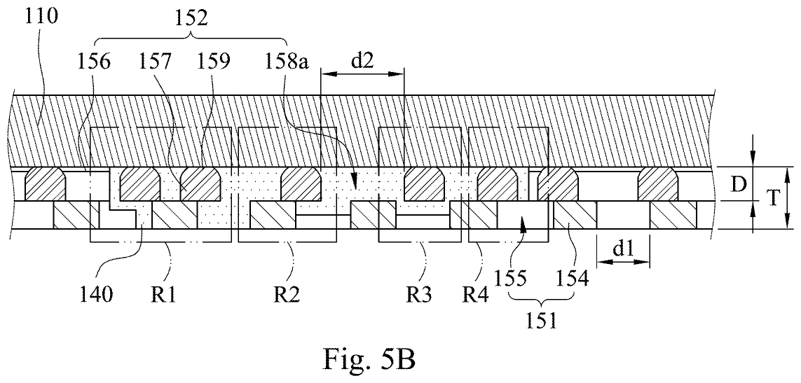

Reference is made to FIG. 5A and FIG. 5B. FIG. 5A is a sectional schematic view along the line 5A-5A of the pad 150 of the physiological signal monitoring device 100 of FIG. 2. FIG. 5B is a partially enlarged schematic view of the region 5B of the physiological signal monitoring device 100 of FIG. 4. In FIG. 5A, the coupling backing 152 fabricated by weaving the plurality of second threads 157 further includes a plurality of piques 159, which are raised due to the weave. The plurality of piques 159 are arranged on the coupling surface 156 to allow the coupling surface 156 having convex and concave three-dimensional textures thereon. The area and directions of combining the adhesive layer 140 and the coupling surface 156 can be increased, which improves the connecting strength between the base 110 and the pad 150. Furthermore, in FIG. 5B, spaces can be formed between the base 110 and the pad 150 because of the convex and concave textures on the coupling surface 156 after the pad 150 and the base 110 are assembled. The air permeability between the base 110 and the pad 150 is improved, that is, it creates the spaces for lateral air flow. Each of the plurality of second holes 158a includes an opening end at the coupling surface 156, and a height H of each of the plurality of piques 159 protruding from each of the opening ends at the coupling surface 156 can be larger than or equal to 0.2 mm and smaller than or equal to 0.5 mm. The connecting strength between the base 110 and the pad 150 can be maintained, and proper sizes of the spaces between the base 110 and the pad 150 can be ensured.

In FIG. 5B, at least part of the adhesive layer 140 soaks the pad 150 through the plurality of second holes 158a of the coupling backing 152 and at least partially wraps at least one of the plurality of second threads 157 and the plurality of first threads 154. For example, as shown in the region R1 of FIG. 5B, at least part of the adhesive layer 140 can completely soak the first holes 155 and wrap the second threads 157 and the first threads 154. As shown in the region R2 of FIG. 5B, at least part of the adhesive layer 140 can soak the first holes 155 and wrap the second threads 157 and part of the first threads 154. As shown in the region R3 of FIG. 5B, when the second threads 157 and the first threads 154 do not overlap, at least part of the adhesive layer 140 can soak the first holes 155 and completely wrap the second threads 157. Furthermore, as shown in the region R4 of FIG. 5B, at least part of the adhesive layer 140 can only soak the second holes 158a and wrap the second threads 157, but not wrap the first threads 154. It should be mentioned that, the four conditions of the regions R1 to R4 are all illustrated in FIG. 5B, but the pad 150 of the present disclosure is not limited to FIG. 5B. The pad 150 of the present disclosure can include at least one of the conditions of the regions R1 to R4. If the adhesive layer 140 more completely wraps the second threads 157 and the first threads 154, the retaining force between the pad 150 and the base 110 can be increased. If the adhesive layer 140 only wraps part of the second threads 157 and the first threads 154, the air permeability between the pad 150 and the base 110 can be maintained.

Moreover, at least one of a portion of the plurality of first threads 154 and a portion of the plurality of first holes 155 of the adhesive backing 151 can be under the plurality of second holes 158a, so as to facilitate the adhesive layer 140 soaking the plurality of first holes 155 from the plurality of second holes 158a and at least partially wrapping the plurality of first threads 154. A diameter d1 of each of the plurality of first holes 155 can be smaller than a diameter d2 of each of the plurality of second holes 158a. When the adhesive layer 140 soaks the adhesive backing 151 from the coupling backing 152, the adhesive layer 140 is less likely passing through the plurality of first holes 155. Thus, it can slow down the adhesive layer 140 moving from the coupling backing 152 to the adhesive backing 151, so as to control the region which the adhesive layer 140 wraps, and prevent the adhesive layer 140 affecting the adhesion between the adhesive surface 153 and the skin surface or prevent the problems such as skin allergy caused by excessed adhesive. Furthermore, in FIG. 4, the coupling backing 152 fabricated by weaving the plurality of second threads 157 can include not only the plurality of second holes 158a, but also a plurality of third holes 158b which are smaller than the diameter d2 of each of the plurality of second holes 158a. The air permeability of the pad 150 can be improved. However, it should be noticed that the present disclosure is not limited thereto.

Even the present disclosure is not limited to any one of the aforementioned embodiments, the present disclosure aims to reach a balance between the retaining force and air permeability between the pad 150 and the base 110. When a thickness of the pad 150 is T, an embedded depth of the adhesive layer 140 and the pad 150 is D, and the condition of 0<D/T≤0.5 can be satisfied. Therefore, the connecting strength between the base 110 and the pad 150 can be ensured. The adhesive layer 140 does not completely soak the pad 150 and a certain degree of air permeability can be maintained, as well as it prevents the adhesive layer 140 touching the skin surface of the organism.

In other embodiments, the pad at least includes an adhering layer and a coupling layer. The adhering layer provides an adhesive surface and includes a plurality of first holes. The pad is attached to the skin surface of the organism through the adhesive surface. The coupling layer provides a coupling surface and includes a plurality of second holes, which are open at the coupling surface. At least part of the adhesive layer soaks the pad through the coupling surface of the coupling layer, so as to enhance the retaining force between the pad and the base. In other embodiments, the material and manufacturing method of the pad are not limited. For example, the adhering layer and the coupling layer with porous structure can be manufactured by injection molding process. Alternatively, the adhering layer and the coupling layer of the pad can be fabricated by weaving a plurality of threads respectively, which makes the pad be similar to a sandwich mesh fabric. The adhesive layer at least partially wraps the plurality of threads, so as to provide great air permeability and flexibility. If the weaving method is adopted, the coupling layer can further include a plurality of piques arranged on the coupling surface, and a relief texture is formed on the coupling surface. The aforementioned piques have the structure similar to the piques 159, the combination of the adhesive layer and the pad is also similar to the aforementioned embodiment, and the details will not be given herein. However, in other embodiments, the relief texture on the coupling surface can be additionally fabricated on the pad. When the adhesive layer soaks the pad through the coupling surface of the coupling layer, the adhesive layer can at least hold the aforementioned relief texture to further enhance the retaining force between the pad and the base.

In addition, the adhering layer and the coupling layer can be directly or indirectly connected to each other. If the adhering layer and the coupling layer are directly connected to each other, it means that the adhering layer and the coupling layer can be connected by gluing, weaving or integrally forming. If the adhering layer and the coupling layer are indirectly connected to each other, there can be other structural layers between the adhering layer and the coupling layer based on the requirements, which is favorable for adjusting the mechanical properties or air permeability of the pad.

No matter in which embodiment, a retaining force of the pad connected to the base can be larger than 10 kgf, and the retaining force of the pad connected to the base can be larger than an adhesion between the pad and the skin surface of the organism. Thus, it is not easy for the pad and the base to be separated. It is further ensured that the pad will not remain on the skin surface when the physiological signal monitoring device is removed from the skin surface of the organism.

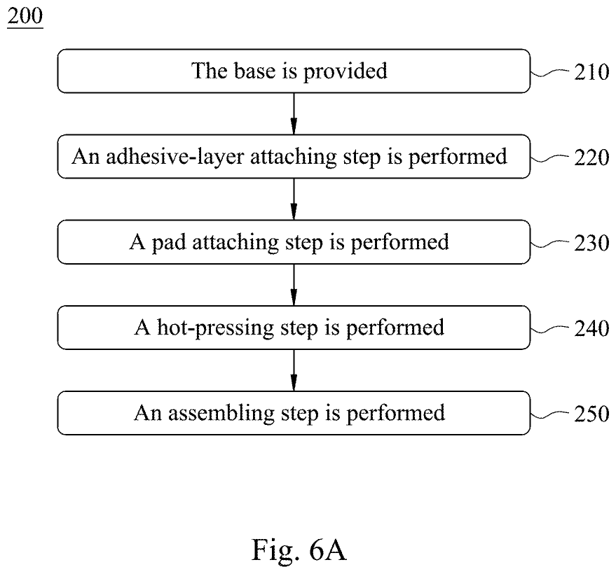

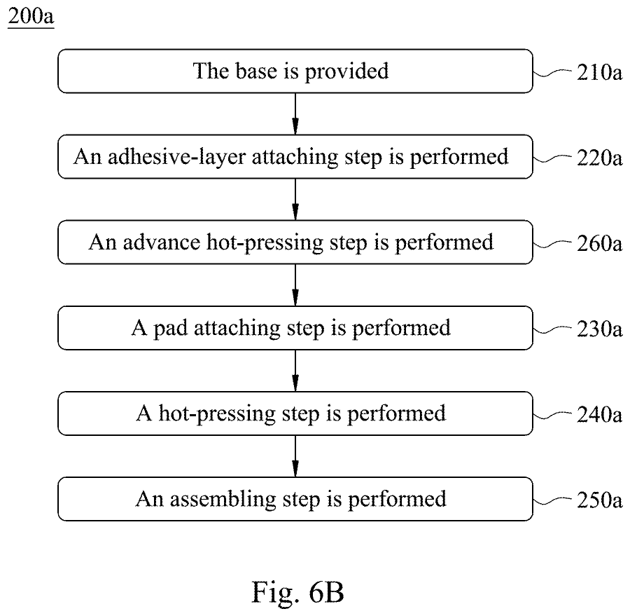

Reference is made to FIG. 2 and FIG. 6A. FIG. 6A is a step flow chart of a method 200 for enhancing the adhesion of the physiological signal monitoring device 100 on the skin surface of the organism according to an embodiment of another aspect of the present disclosure. The method 200 for enhancing the adhesion of the aforementioned physiological signal monitoring device 100 on the skin surface of the organism includes Step 210, Step 220, Step 230, Step 240 and Step 250.

In Step 210, the base 110 is provided. The details of the base 110 are introduced in the aforementioned paragraphs, which will not be given herein.

In Step 220, an adhesive-layer attaching step is performed to attach the adhesive layer 140 to the bottom surface of the base 110. The adhesive layer 140 can be formed by coating a liquid adhesive on the bottom surface of the base 110, or by attaching a solid adhesive film on the bottom surface of the base 110, which the present disclosure is not limited to.

In Step 230, a pad attaching step is performed to attach the pad 150 to the adhesive layer 140. That is, the pad 150 is attached to the adhesive layer 140 with the coupling surface 156 toward the adhesive layer 140.

In Step 240, a hot-pressing step is performed by hot pressing from the pad 150 toward the base 110, so as to make the pad 150 be connected to the base 110 through the adhesive layer 140. A temperature of hot pressing can be 115° C. to 125° C., a pressure thereof can be 3.5 kg/cm² to 4.5 kg/cm², and it can be hot pressed for 10 seconds to 20 seconds. Therefore, the degree, such as the depth and range, of the adhesive layer 140 soaking the pad 150 can be controlled, which prevents the adhesive layer 140 from over-soaking the pad 150 and affecting the air permeability.

In Step 250, an assembling step is performed. When a user uses the physiological signal monitoring device 100 to monitor the analyte in the organism, the transmitter 130 and the sensor 120 are assembled on the base 110 which has been hot pressed, so as to finish setting up the physiological signal monitoring device 100. Because the transmitter 130 and the sensor 120 are assembled on the base 110 after hot pressing, it can prevent the high temperature of hot pressing affects the precision elements such as the transmitter 130 and the sensor 120.

Reference is made to FIG. 6B. FIG. 6B is a step flow chart of the method 200a for enhancing the adhesion of the physiological signal monitoring device 100 on the skin surface of the organism according to another embodiment of another aspect of the present disclosure. The method 200a for enhancing the adhesion of the aforementioned physiological signal monitoring device 100 on the skin surface of the organism includes Step 210a, Step 220a, Step 230a, Step 240a, Step 250a and Step 260a. Step 210a, Step 220a, Step 230a, Step 240a and Step 250a are respectively the same as Step 210, Step 220, Step 230, Step 240 and Step 250 in the aforementioned method 200, and the details will not be given herein.

In Step 260a, an advance hot-pressing step is performed before attaching the pad 150 in Step 230a. The advance hot-pressing step is performed by hot pressing from the adhesive layer 140 toward the base 110, so as to make the adhesive layer 140 be connected to the base 110 first. A temperature of advance hot pressing can be 75° C. to 85° C., a pressure thereof can be 3.5 kg/cm² to 4.5 kg/cm², and it can be advance hot pressed for 3 seconds to 10 seconds. The combination between the adhesive layer 140 and the base 110 can be improved by the advance hot-pressing step, so as to enhance the retaining force between the pad 150 and the base 110.

The retaining force and adhesion of the physiological signal monitoring device according to the present disclosure are tested as follows.

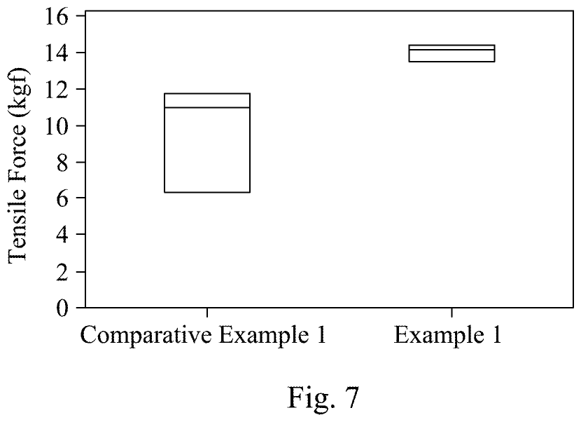

Reference is made to FIG. 7. FIG. 7 is a test diagram showing the retaining force between the pad and the base of Comparative Example 1 and Example 1. The physiological signal monitoring device of Comparative Example 1 uses a double-sided tape as the adhesive layer, and the physiological signal monitoring device of Example 1 uses thermoplastic polyurethane as the adhesive layer. In this test, a cotton rope is tied at the pad, and the pad is pulled with the cotton rope after the base is firmly set. The tensile force of the pad being separated from the base is recorded.

In FIG. 7, the tensile force of Comparative Example 1 is about 10 kgf, while the tensile force of Example 1 is about 14 kgf. It should be mentioned that, the tensile force of Example 1 is the force when the cotton rope breaks. When the cotton rope breaks, the pad and the base of Example 1 are not separated yet, which means the real retaining force of Example 1 should be much higher than 14 kgf. From the data of tensile force, it can be understood that the pad and the base of the physiological signal monitoring device of the present disclosure are connected relatively firmly. The problem of the base separated from the pad prematurely as wearing the physiological signal monitoring device can be effectively prevented.

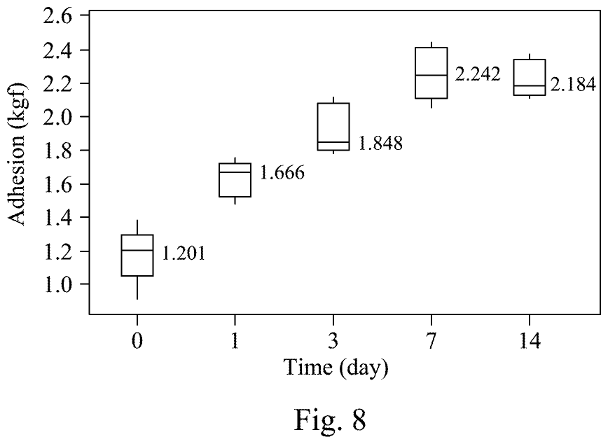

Reference is made to FIG. 8. FIG. 8 is a simulating test diagram showing the adhesion between the pad and the skin surface of the organism of Example 2. In this test, the pad, which is first combined with the base, is pressed to a steel board with a pressure of 2 kg for 30 seconds. The pad with the steel board stays in an environment with ambient temperature and ambient humidity for 1 day, 3 days, 7 days or 14 days. Then, the adhesion between the pad and the steel board is measured, so as to simulate the change in adhesion caused by the physiological signal monitoring device attaching to the skin surface of the organism for a long time.

In FIG. 8, the adhesion between the pad and the steel board of Example 2 is increased over time, and reaches the highest adhesion (about 2.2 kgf) around the seventh day. It means that the physiological signal monitoring device of the present disclosure still remains great adhesion and is less likely to fall off after attaching to the skin surface of the organism for a long time. Also, by comparing to the data of FIG. 7, the retaining force between the pad and the base is larger than the adhesion between the pad and the skin surface of the organism, the pad can be removed with the base without remaining on the skin surface of the organism as removing the physiological signal monitoring device.

In this regard, according to the physiological signal monitoring device of the present disclosure, the pad includes the plurality of first holes and the plurality of second holes to make the adhesive layer soak the pad. The area and directions of combining the adhesive layer and the coupling surface are increased by designing the structure of the coupling surface, which improves the connecting strength between the base and the pad.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A physiological signal monitoring device, which is used for monitoring at least one analyte in an organism, comprising:
a base, wherein the base is configured to be arranged on a skin surface of the organism, and the base comprises a bottom surface;
a sensor carried by the base, wherein the sensor is at least partially implanted under the skin surface of the organism, so as to measure and output a physiological signal corresponding to the analyte;
a transmitter detachably disposed on the base, wherein the transmitter is coupled to the sensor for receiving and transmitting the physiological signal;
an adhesive layer, wherein the adhesive layer is arranged on the bottom surface of the base; and
a pad comprising:
an adhesive backing providing an adhesive surface, wherein the adhesive backing is fabricated by weaving a plurality of first threads and comprises a plurality of first holes; and
a coupling backing providing a coupling surface, wherein the coupling backing is stacked on the adhesive backing and fabricated by weaving a plurality of second threads to comprise a plurality of second holes and a plurality of piques, the plurality of second holes are at least open at the coupling surface, and the plurality of piques are arranged on the coupling surface to form convex and concave three-dimensional textures on the coupling surface;
wherein the pad is attached to the skin surface of the organism through the adhesive surface, and at least part of the adhesive layer soaks the pad through the plurality of second holes of the coupling backing and at least partially wraps at least one of the plurality of second threads and the plurality of first threads, so as to make the pad be connected to the base through the adhesive layer.

2. The physiological signal monitoring device of claim 1, wherein each of the plurality of second holes comprises an opening end at the coupling surface, and a height of each of the plurality of piques protruding from each of the opening ends at the coupling surface is larger than or equal to 0.2 mm and smaller than or equal to 0.5 mm.

3. The physiological signal monitoring device of claim 1, wherein a diameter of each of the plurality of first holes is smaller than a diameter of each of the plurality of second holes.

4. The physiological signal monitoring device of claim 1, wherein at least one of a portion of the plurality of first threads and a portion of the plurality of first holes of the adhesive backing is under the plurality of second holes.

5. The physiological signal monitoring device of claim 1, wherein a thickness of the pad is T, an embedded depth of the adhesive layer and the pad is D, and the condition of 0<D/T≤0.5 is satisfied.

6. A physiological signal monitoring device, which is used for monitoring at least one analyte in an organism, comprising:
a base, wherein the base is configured to be arranged on a skin surface of the organism, and the base comprises a bottom surface;
a sensor carried by the base, wherein the sensor is at least partially implanted under the skin surface of the organism, so as to measure and output a physiological signal corresponding to the analyte;
a transmitter detachably disposed on the base, wherein the transmitter is coupled to the sensor for receiving and transmitting the physiological signal;
an adhesive layer, wherein the adhesive layer is arranged on the bottom surface of the base; and
a pad at least comprising an adhering layer and a coupling layer;
wherein the adhering layer provides an adhesive surface and comprises a plurality of first holes, and the coupling layer provides a coupling surface and comprises a plurality of second holes, which are open at the coupling surface;
wherein the pad is attached to the skin surface of the organism through the adhesive surface, and at least part of the adhesive layer soaks the pad through the coupling surface of the coupling layer, so as to make the pad be connected to the base through the adhesive layer.

7. The physiological signal monitoring device of claim 6, wherein the coupling layer further comprises a plurality of piques arranged on the coupling surface, and a relief texture is formed on the coupling surface.

8. The physiological signal monitoring device of claim 6, wherein the adhering layer and the coupling layer are directly or indirectly connected to each other.

9. The physiological signal monitoring device of claim 6, wherein the adhering layer and the coupling layer of the pad are fabricated by weaving a plurality of threads respectively, when the adhesive layer soaks the pad through the coupling surface of the coupling layer, the adhesive layer at least partially wraps the plurality of threads.

10. The physiological signal monitoring device of claim 6, wherein the bottom surface of the base is in a rectangular shape, and the adhesive layer is arranged at four corners of the bottom surface.

11. The physiological signal monitoring device of claim 6, wherein the adhesive layer is arranged on the bottom surface in a U shape.

12. The physiological signal monitoring device of claim 6, wherein a material of the adhesive layer is thermoplastic polyurethane.

13. The physiological signal monitoring device of claim 6, wherein a retaining force of the pad connected to the base is larger than 10 kgf.

14. The physiological signal monitoring device of claim 6, wherein a retaining force of the pad connected to the base is larger than an adhesion between the pad and the skin surface of the organism.

15. A method for enhancing an adhesion of the physiological signal monitoring device of claim 1 or claim 6 on a skin surface of an organism, comprising:
providing the base;
performing an adhesive-layer attaching step to attach the adhesive layer to the bottom surface of the base;
performing a pad attaching step to attach the pad to the adhesive layer;
performing a hot-pressing step by hot pressing from the pad toward the base, so as to make the pad be connected to the base through the adhesive layer; and
performing an assembling step, when a user uses the physiological signal monitoring device to monitor the analyte in the organism, the transmitter and the sensor are assembled on the base which has been hot pressed, so as to finish setting up the physiological signal monitoring device.

16. The method of claim 15, wherein performing the hot-pressing step is to hot press under a temperature of 115° C. to 125° C. and a pressure of 3.5 kg/cm² to 4.5 kg/cm² for 10 seconds to 20 seconds.

17. The method of claim 15, further comprising performing an advance hot-pressing step before performing the pad attaching step, wherein the advance hot-pressing step is performed by hot pressing from the adhesive layer toward the base, so as to make the adhesive layer be connected to the base.

18. The method of claim 17, wherein performing the advance hot-pressing step is to hot press under a temperature of 75° C. to 85° C. and a pressure of 3.5 kg/cm² to 4.5 kg/cm² for 3 seconds to 10 seconds.
